(19) United States
(12) Patent Application Publication
(10) Pub. No.: US 2011/0208292 A1
(43) Pub. Date: Aug. 25, 2011

(54) HINGED SHEATH ASSEMBLY AND METHOD OF USE

(75) Inventors: Randolf Von Oepen, Los Altos, CA (US); Travis R. Yribarren, Campbell, CA (US)

(73) Assignee: ABBOTT LABORATORIES, Santa Clara, CA (US)

(21) Appl. No.: 12/708,666

(22) Filed: Feb. 19, 2010

(51) Int. Cl. A61F 2/84

(52) U.S. Cl. 623/1.23

(57) ABSTRACT

A sheath assembly is provided for protecting a stent mounted on a catheter. An inner tubular member is positioned over the stent without longitudinal movement of the inner tubular member along the stent surface thereby eliminating the possibility of scraping or scratching a drug coating or polymer coating on the stent surface. An outer tubular member slides over the inner tubular member to firmly compress it onto the stent for further protection. In use, the outer tubular member is removed from over the inner tubular member so that the inner tubular member can open similar to a clamshell opening radially outwardly away from the stent without longitudinal movement along the stent surface.

### BACKGROUND

The invention relates to stent delivery systems, which are used to implant a stent into a patient's body lumen to maintain the patency thereof. More particularly, the present invention relates to a hinged sheath assembly that is mounted over a stent on a catheter for deploying the stent in a body lumen.

Stents are generally cylindrically-shaped devices which function to hold open and sometimes expand a segment of a blood vessel or other body lumen such as a coronary artery. They are also suitable to support and hold back a dissected arterial lining that can occlude the fluid passageway. Stents also are useful in maintaining the patency of a body lumen, such as a coronary artery, after a percutaneous transluminal coronary angioplasty (PTCA) procedure. The delivery and deployment of stents in coronary arteries are well known in the art and various types of catheters are used, along with guidewires, to position and implant the stent in the artery.

Presently, stents are coated with polymer coatings that may include a drug for the purpose of reducing the likelihood of the development of restenosis. The polymer coating and the drug can be easily scratched or removed if the drug coating comes into contact with any outside agency. It is important to maintain the integrity of the stent surface in order to ensure the clinical efficacy of the drug coating. Traditionally, in order to protect the stent surface, a sheath is applied over the stent, which is then packaged until it is removed from the package by the physician in preparation for use. When the sheath is applied to or removed from the stent, it slides over the stent and may cause damage to the polymer coating or the drug coating.

It is therefore important to improve the existing sheath assemblies to protect the polymer and drug coatings on the stent during both the application and removal of the sheath assembly from the stent surface. The present invention satisfies these needs.

### SUMMARY OF THE INVENTION

The present invention is directed to a sheath assembly for protecting a stent mounted on a catheter. In one embodiment, the sheath assembly has an outer tubular member and an inner tubular member where the inner tubular member has at least two longitudinal slits that extend from a proximal end of the inner tubular member to a point near the distal end of the inner tubular member. The inner tubular member is hinged where the longitudinal slit terminates near the distal end of the inner tubular member. Thus, the inner tubular member has a clamshell-type configuration so that it can essentially open radially outwardly and close radially inwardly onto the stent mounted on a catheter, without the possibility of sliding longitudinally along the stent thereby preventing any scraping or scratching movement on the stent surface, and specifically on the drug coated polymer coating on the stent surface. The inner tubular member is positioned over the stent by moving the generally half-cylindrical portions formed by the longitudinal slits radially inwardly to in essence clamp down on the stent. Thereafter, the outer tubular member slides over the inner tubular member in a tight-fitting manner so as to slightly compress the inner tubular member onto the stent. The entire catheter assembly, including the stent and the protective sheath assembly, are packaged in a known manner. In use, the physician removes the catheter assembly from the packaging, and pulls the outer tubular member in a distal direction to slide it distally off of the inner tubular member. Thereafter, the inner tubular member can be removed from the stent by gently squeezing on the distal end of the inner tubular member at the hinged portion, thereby causing the half-cylindrical portions of the inner tubular member to move radially outwardly away from the stent without any longitudinal movement along the stent surface.

In another embodiment, the protective sheath assembly has the same basic configuration as described above, however, the hinge portion includes a radiused curve positioned at the distal end of the longitudinal slits. The radiused curve has a keyhole configuration that functions as a hinge to permit the half-cylindrical portions of the inner tubular member to open and close in a radial direction, much like a clamshell opens and closes.

In another embodiment, the protective sheath assembly has the same basic configuration as described above, however, the inner tubular member has an outer ridge near its distal end, and the outer tubular member has an inner ridge near its proximal end thereby creating an interference between the ridges so that the sheath assembly remains a unitary assembly. More specifically, as the outer tubular member is moved distally over the inner tubular member, the inner ridge of the outer tubular member engages the outer ridge on the inner tubular member so that both the inner tubular member and outer tubular member stay connected as the sheath assembly is removed from the stent.

Other features and advantages of the present invention will become more apparent from the following detailed description of the invention when taken in conjunction with the accompanying drawings.

### BRIEF DESCRIPTION OF THE DRAWINGS

FIG. 1 is a side view, partially in section, depicting a delivery catheter and stent, with a prior art sheath covering the stent.

FIG. 2 is a side view of the sheath assembly including the inner tubular member having longitudinal slits and a hinge portion and being covered by the outer tubular member.

FIG. 3 is a side view, partially in section, depicting the delivery catheter with the sheath assembly mounted on the catheter and the outer tubular member being partially removed from over the inner tubular member.

FIG. 4 is a side view, partially in section, depicting the catheter assembly including the outer tubular member being completely removed from over the inner tubular member and the inner tubular member being expanded radially outwardly away from the stent mounted on the catheter.

FIG. 5 is a side view, partially in section, showing the catheter assembly with the stent mounted thereon and the inner tubular member and outer tubular member being completely removed from the catheter assembly.

FIG. 6 is a side view, partially in section, depicting a ridge on the inner surface of the outer tubular member and a ridge on the outer surface of the inner tubular member which engage in order to hold the inner and outer tubular members together.

FIG. 7 is a side view, partially in section, depicting an outer tubular member and inner tubular member that is a unitary structure for mounting on a stent catheter assembly.

### DETAILED DESCRIPTION OF THE PREFERRED EMBODIMENTS

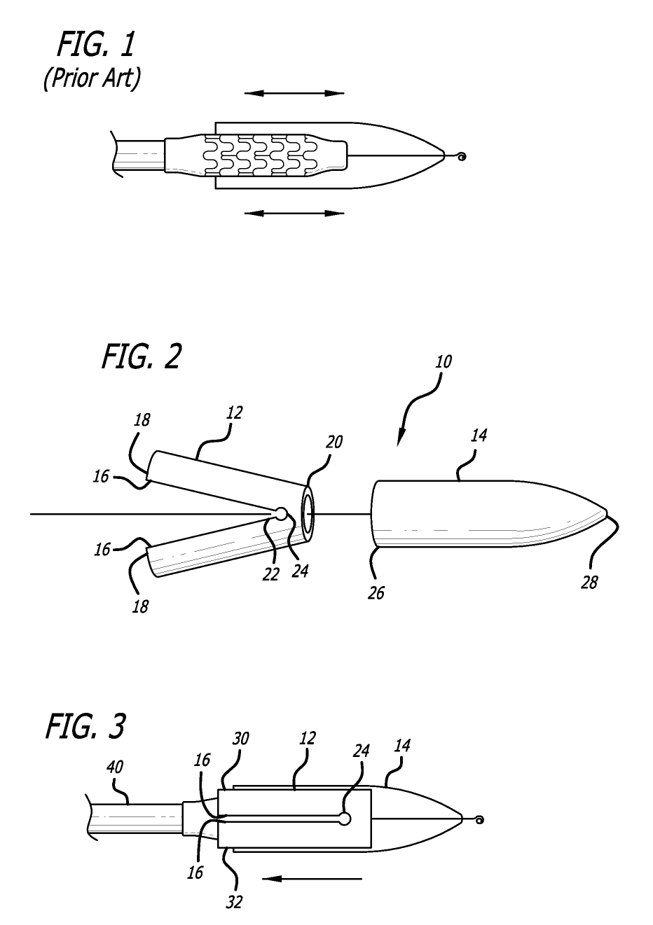

Prior art stent delivery systems, such as the one shown in FIG. 1, typically have an intravascular stent mounted on a balloon catheter with a removable sheath covering the stent for protection. The protective sheath slides longitudinally over the stent for protecting the stent during packaging and delivery to the end user. When the physician is ready to deploy the stent, the sheath is removed by pulling it longitudinally off of the stent in a distal direction. Since the protective sheath fits tightly on the stent in order to protect it, it can actually scratch or scrape along the stent surface and cause damage to the stent. This is particularly damaging when the stent is coated with a therapeutic agent or drug which may also include a polymer which is infused with the therapeutic agent. If the drug or therapeutic agent is scratched or scraped when the protective sheath is placed over the stent or removed from the stent, it can adversely affect the efficacy of the drug eluting from the stent when it is deployed in a coronary artery. The present invention solves the problem of sliding the protective sheath on and off the catheter assembly so that the drug coating on the stent is not scratched or scraped during the process.

Still referring to FIG. 1, a portion of a prior art balloon catheter is depicted in which a stent is mounted on the catheter with a conventional sheath covering the stent. In the prior art devices such as the one shown in FIG. 1, the sheath fits firmly over the stent and moves in a longitudinal direction to slide over the stent. Thus, to protect the stent during packaging, the sheath is pushed over the distal end of the catheter so that the sheath slides in a proximal direction over the stent. The entire catheter assembly along with the sheath is packaged for storage and eventually delivery to a physician. After the catheter assembly is removed from the packaging, the physician removes the sheath by sliding it longitudinally in a distal direction off of the stent so that the stent is exposed for eventual deployment in an artery. Sliding the sheath on and off the stent can scrape or scratch the drug coating or polymer matrix that is coated onto the stent.

In keeping with the present invention, as shown in FIGS. 2-5, a sheath assembly 10 is provided for protecting a stent mounted on a catheter and to avoid the longitudinal movement of a sheath sliding over the stent surface. More particularly, sheath assembly 10 includes an inner tubular member 12 and an outer tubular member 14. The inner tubular member 12 fits over a stent and the outer tubular member slidingly engages and extends over the inner tubular member 12. As shown in FIG. 2, the inner tubular member 12 has a pair of longitudinal slits 16 extending from the proximal end 18 of the inner tubular member toward the distal end 20 of the inner tubular member. The longitudinal slits 16 terminate at a point 22 that is near the distal end 20 of the inner tubular member 12, but not at the distal end. Further, a hinge portion 24 is formed at the terminal distal end of the longitudinal slits at point 22. The outer tubular member 14, which is configured to slide over the inner tubular member, has a proximal end 26 and a distal end 28. The longitudinal slits 16 in the inner tubular member result in a first half-cylindrical portion 30 and a second half-cylindrical portion 32. In conjunction with the hinge portion 24, the first half-cylindrical portion 30 and the second half-cylindrical portion 32 open and close similar to a clamshell opening and closing. Importantly, the first half-cylindrical portion 30 and the second half-cylindrical portion 32 move radially outwardly or inwardly so that there is no longitudinal movement of the inner tubular member on the surface of the stent, thereby removing any likelihood that the inner tubular member will scratch or scrape the drug or polymer surface on the stent. While the longitudinal slits 16 can be diametrically opposed resulting in the first and second half-cylindrical portions 30,32, the longitudinal slits can be positioned on the circumference of the inner tubular member 12 so that the resulting arcuate portions are not precisely 180° arcuate portions like the half-cylindrical portions 30,32.

With reference to FIG. 3, the sheath assembly 10 is mounted on the distal portion of a balloon catheter 40. Only the distal portion of the balloon catheter 40 is shown, as balloon catheters are well known in the art and can have numerous configurations. A stent 42 is mounted on the balloon portion of the balloon catheter, but the stent 42 is not visible since it is covered by the inner tubular member 12. Still referring to FIG. 3, the inner tubular member 12 has been placed over the stent 42 by moving the first half-cylindrical portion 30 and the second half-cylindrical portion 42 radially inwardly until they are firmly positioned over the stent 42. As set forth above, the hinge portion 24 permits the first and second half-cylindrical portion 30,32 to move in a clamshell-like movement in order to open and close onto the stent. Further, the outer tubular member 14 is pushed over the inner tubular member 12 after it is positioned over the stent. The outer tubular member fits snugly onto the inner tubular member so that it compresses the inner tubular member onto the stent 42. Although the outer tubular member 14 slides over the inner tubular member 12, this does not cause any scraping or scratching on the drug coating on the stent since the inner tubular member 12 covers and protects the stent from the longitudinal movement as the outer tubular member slides over the inner tubular member.

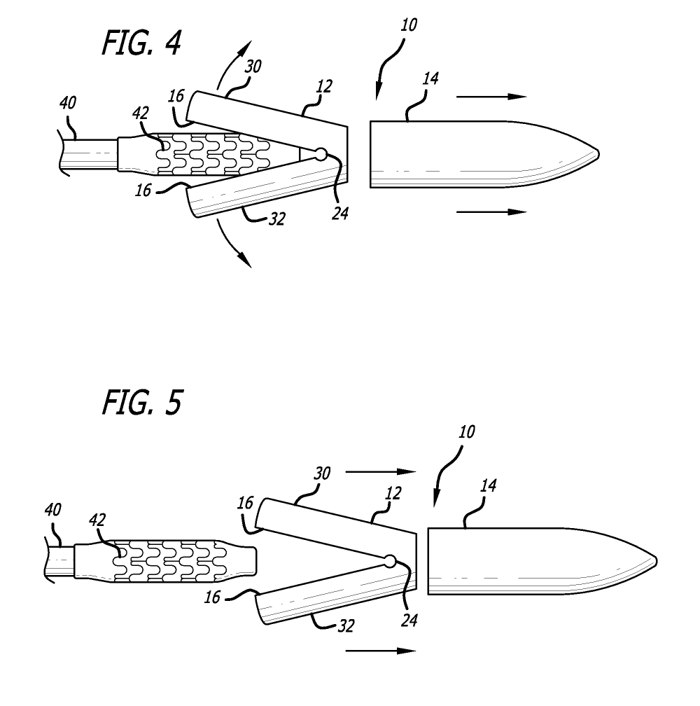

As can be seen in FIGS. 4 and 5, the sheath assembly 10 is being removed from the balloon catheter 40 and stent 42. The outer tubular member 14 is withdrawn distally from over the inner tubular member 12. After the outer tubular member 14 is completely withdrawn from the inner tubular member 12, the physician can gently squeeze on the hinge portion 24 thereby causing the first half-cylindrical portion 30 and the second half-cylindrical portion 32 to move in a clamshell-like movement radially outwardly away from the stent 42. After the inner tubular member 12 is completely clear of the stent, it can be removed from the balloon catheter 40 by withdrawing the inner tubular member 12 distally as shown in FIG. 5. Importantly, as the first half-cylindrical portion 30 and the second half-cylindrical portion 42 move radially outwardly away from the stent, they do not scrape or scratch the stent surface or any drug coating or polymer coating on the stent surface.

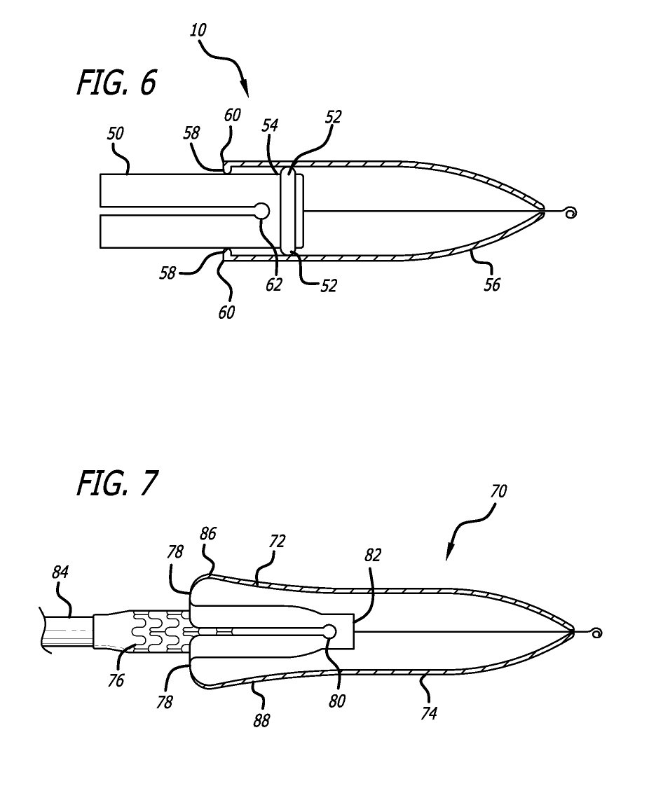

In another embodiment, as shown in FIG. 6, the sheath assembly 10 is configured so that it remains as a single unit after it is removed from the balloon catheter assembly. More specifically, in this embodiment an inner tubular member 50 is substantially the same as the previously described inner tubular member 12 with the exception of ridges. Inner tubular member 50 has an outer ridge 52 that extends circumferentially around the distal portion 54 of the inner tubular member 50. The outer tubular member 56 has an inner ridge 58 that extends circumferentially at or near the proximal end 60 of the outer tubular member 56. In this embodiment, as the outer tubular member 56 is withdrawn distally over the inner tubular member 50, the inner ridge 58 will engage the outer ridge 52 thereby preventing the separation of the inner tubular member 50 from the outer tubular member 56. The outer ridge and the inner ridge are positioned so that they do not interfere with the operation of hinge portion 62 thereby allowing the inner tubular member to open and close as previously described.

Referring to FIG. 7, another embodiment of sheath assembly 70 is shown. In this embodiment, the inner tubular member 72 and the outer tubular member 74 are a single structure that can be moved over the stent 76 without causing a longitudinal or sliding movement along the stent surface. The outer tubular member 74 is formed from a polymer material that is substantially more rigid and configured to have the column strength to slide over the inner tubular member. The inner tubular member 72 is made of a softer elastomeric material that is flexible enough to roll along the surface of stent 76 as the more rigid outer tubular member 74 is pushed in a proximal direction over the inner tubular member 72. The inner tubular member 72 has longitudinal slits 78 that extend a substantial portion along the length of the inner tubular member 72. A hinge portion 80 is at the distal end of the longitudinal slits 78. In this embodiment, the distal end 82 of the inner tubular member 72 is also formed of a softer elastomeric material that can grip onto the distal end of the catheter 84. As the outer tubular member 74 is pushed in a proximal direction, the distal end 82 grips onto the distal end of the catheter 84 and the first half-cylindrical portion 86 and the second half-cylindrical portion 88 roll onto the stent and the balloon portion of the catheter 84. As the first and second half-cylindrical portions 86,88 roll onto the stent, there is no longitudinal movement of the outer tubular member relative to the stent, thereby eliminating the possibility that the inner tubular member will scrape or scratch the drug coating on stent 76. As the outer tubular member 74 slides over the inner tubular member 72 it slightly compresses the inner tubular member onto the stent 76 thereby protecting the stent from any outside agency. The process by which the sheath assembly 70 is removed from the catheter 84 is the reverse of that shown in FIG. 7. As the outer tubular member 75 slides in a distal direction away from the catheter 84, the distal end 82 of the inner tubular member 72 grips the distal end of the catheter 84 thereby allowing the first and second half cylindrical portions 86 and 88 to roll off of the stent radially outwardly as the inner tubular member inverts. This insures that there is no axial movement that can scrape or damage the stent surface or any drug coating or polymer coating on the stent surface. Eventually, the inner tubular member 72 will invert completely and the sheath assembly 70 can be removed from the catheter.

The sheath assembly embodiments shown in FIGS. 2-7 can be formed from a number of polymer materials that are well known in the art. Depending upon the application, either of the inner and outer tubular members may be flexible or more rigid and be formed from polyethylene materials such as linear low density polyethylene (LLDPE), high density polyethylene (HDPE) or low density polyethylene (LDPE), as examples. For example, the inner tubular members can be formed from an elastomeric polymer such as urethane, rubber, latex and TECOFLEX®. The outer tubular member preferably is formed of a more rigid material than the inner tubular member and can be formed of such materials as polyether ether ketone (PEEK), a rigid plastic such as acrylonitrile-butadiane-styrene (ABS) or polyvinyl chloride (PVC) or from a fiber or braid reinforced tube from any combination of these materials, all of which will enhance the pushability of the outer tubular member over the inner tubular member.

Although preferred alternative embodiments have been described and illustrated, the invention is susceptible to modifications and adaptations within the ability of those skilled in the art and without the exercise of inventive faculty. Thus, it should be understood that various changes in form, detail, and usage of the present invention may be made without departing from the spirit and scope of the invention. Accordingly, it is not intended that the invention be limited, except as by the appended claims.

What is claimed:

1. A sheath assembly for protecting a stent mounted on a catheter, comprising:
a sheath assembly having an outer tubular member and an inner tubular member;
the inner tubular member having at least two longitudinal slits; and
the outer tubular member extending over at least a portion of the inner tubular member.

2. The sheath assembly of claim 1, wherein the outer tubular member is configured for slidable movement over the inner tubular member.

3. The sheath assembly of claim 2, wherein the outer tubular member has an inner diameter and an outer diameter and the inner tubular member has an inner diameter and an outer diameter, the inner diameter of the outer tubular member being greater than the outer diameter of the inner tubular member.

4. The sheath assembly of claim 2, wherein the outer tubular member is sized to compress the inner tubular member when the outer tubular member slides over the inner tubular member.

5. The sheath assembly of claim 1, wherein the outer tubular member is formed from a rigid polymer material and the inner tubular member is formed from a polymer material including urethane, rubber, latex, LLDPE, HDPE and LDPE.

6. The sheath assembly of claim 1, wherein the at least two longitudinal slits in the inner tubular member produce two generally half-cylindrical portions.

7. The sheath assembly of claim 6, wherein the at least two longitudinal slits extend along a substantial portion of the inner tubular member.

8. The sheath assembly of claim 7, wherein the inner tubular member has a hinge portion at a distal end of the longitudinal slits so that the two generally half-cylindrical portions can open and close in a clamshell configuration.

9. The sheath assembly of claim 8, wherein the hinge portion has a keyhole configuration.

10. The sheath assembly of claim 1, wherein the inner tubular member has an outer ridge and the outer tubular member has an inner ridge thereby creating an interference between the ridges so that the sheath assembly remains a unitary assembly.

11. A sheath assembly for protecting a stent mounted on a catheter, comprising:
a sheath assembly having an outer tubular member and an inner tubular member;
the inner tubular member having two longitudinal slits generally diametrically opposed; and
the outer tubular member configured for slidable movement over the inner tubular member so that the outer tubular member applies a slight compressive force on the inner tubular member.
